(19) United States
(12) Patent Application Publication
(10) Pub. No.: US 2006/0116305 A1
(43) Pub. Date: Jun. 1, 2006

(54) Cleansing composition

(75) Inventors: Naoya Yamato, Kawasaki-shi (JP); Masatoshi Saito, Kawasaki-shi (JP); Eiko Oshimura, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 11/267,504

(22) Filed: Nov. 7, 2005

Related U.S. Application Data
(63) Continuation of application No. PCT/JP04/06116, filed on Apr. 28, 2004.

(30) Foreign Application Priority Data
May 7, 2003 (JP) 129539/2003

(51) Int. Cl. C11D 17/00; A61K 8/73

(52) U.S. Cl. 510/124; 510/424; 510/499

(57) ABSTRACT

The present invention provides a cleansing composition for the skin or hair, which contains, together with (A) a sulfate type anionic surfactant and (B) an N-acylalanine or a salt thereof, at least one kind of compound selected from (C) an amphoteric surfactant, (D) a cationic polymer, (E) a silicone compound and (F) an oily material, wherein the mixing ratio of (A) and (B) of (A)/(B)=99/1-70/30 (wt %) and the concentration of ingredient (A) or (C) is the highest of all the surfactant components in the composition.

### CROSS-REFERENCE TO RELATED APPLICATIONS

The present application is a continuation of PCT/JP2004/006116, filed on Apr. 28, 2004, which claims priority to Japanese Application No. JP 2003-129539, filed on May 7, 2003, which are hereby incorporated by reference in their entirety.

### BACKGROUND OF THE INVENTION

1. Field of the Invention

The present invention provides a cleansing composition comprising, as essential components, a sulfate type anionic surfactant, N-acyl-α-alanine or a salt thereof, and one or more kinds of compounds selected from an amphoteric surfactant, a cationic polymer, a silicone compound and an oily material. More particularly, the present invention relates to a cleansing composition for skin and/or hair, which lathers quickly during cleansing, is superior in lather amount, lather spreading, and lather retention, is free of a residual feeling or a frictional feeling on the skin or hair during rinsing, and gives a moist feeling and a clarifying feeling after drying when used for the skin and a moist feeling and simultaneous softness and lightness when used for the hair.

2. Discussion of the Background

Conventionally, body cleansing agents for the skin employ cleansing components such as anionic surfactants represented by long chain fatty acid or a salt thereof (i.e., higher fatty acid soaps) and betaine-type amphoteric surfactants due to their high latherability and high cleansing performance. However, such body cleansing agents are defective in that they exhibit inferior lather quality, water rinsing performance and, with respect to feeling after drying, these body cleansing agents leave a residual feeling and a frictional feeling upon the skin during rinsing, and a dried-out feeling to the skin after drying. In addition, these compositions are defective in that they leave a residual feeling as well as a frictional feeling to the hair during rinsing, and a dried-out feeling and unruly hair after drying, and the like.

Cationic polymer conditioners such as cationic cellulose have been as a cleansing agent to reduce friction during rinsing, imparting a conditioning effect and the like. However, when contained in a sufficiently large amount so as to afford the aforementioned effect, cationic polymers problematically degrade lather quality and leave a residual feeling during rinsing and after drying.

Silicone compounds have been used in shampoo to give glossy hair, smooth hair and the like. However, when present in the large amounts that are required to afford a sufficient effect, silicone compounds problematically degrade lather quality and water rinsing performance during cleansing, and leave a dried-out feeling to the hair after drying and make hair spread.

While various fats and oils are used for cleansing agents (cleansing composition) to provide a moist feeling to the skin and/or hair, particularly after drying, they are mostly washed away during cleansing. Therefore, it is difficult to give a moist feeling to the skin and/or hair after drying. Moreover, when contained in the large amounts to afford a sufficient effect, fats and oils problematically degrade lather quality.

While glycerol, propylene glycol, sodium lactate and the like are known as moisturizers, body cleansing agents that simply combine these moisturizers with cleansing agents do not sufficiently improve a moist feeling of the skin.

JP-A-09-506351 discloses a cleansing composition containing polyoxyethylene alkyl ether sulfate, an amphoteric surfactant, N-acylsarcosinate and cationic cellulose. However, this cleansing composition is associated with problems in that a residual feeling remains during rinsing of the skin and/or hair, and a moist feeling cannot be obtained after drying. Furthermore, U.S. Pat. No. 5,866,110 and U.S. Pat. No. 5,908,617 disclose cleansing compositions obtained by adding a higher alcohol to the above-mentioned composition disclosed in JP-A-09-506351. However, this composition has associated problems in that the residual feeling during rinsing is not improved, a moist feeling after drying is improved somewhat but a clarifying feeling is inferior, and softness and lightness of hair is difficult to achieve.

JP-A-11-189515 and JP-A-11-29446 disclose shampoo compositions containing polyoxyethylene alkyl ether sulfate, an amphoteric surfactant, a compound selected from N-acylsarcosinate, N-acyl-N-methyl-β-alanine salt, N-acyl glutamate salt and the like, and methylpolysiloxane. However, this composition has associated problems in that it has an inferior frictional feeling of hair during rinsing, and softness and lightness of hair after drying.

In view of the foregoing, there is a critical need for the development of a cleansing composition for skin and/or hair, which lathers quickly during cleansing, is superior in the basic performance of cleansing agents of lather amount, lather spreading, and lather retention, is free of a residual feeling or a frictional feeling during rinsing, and gives, after drying, a moist feeling and a clarifying feeling after use to the skin and a moist feeling and simultaneous softness and lightness to the hair.

### SUMMARY OF THE INVENTION

The present inventors have conducted intensive studies in an attempt to achieve the above-mentioned object and found that the above-mentioned object can be achieved by adding (A) a sulfate type anionic surfactant, (B) N-acyl-α-alanine or a salt thereof and at least one kind of compound selected from the group consisting of (C) an amphoteric surfactant, (D) a cationic polymer, (E) a silicone compound and (F) an oily material, and adjusting the mixing ratio thereof to fall within a particular range.

Accordingly, it is an object of the present invention to provide:

(1) A cleansing composition for skin or hair, which contains the following (A), (B) and (C), wherein the mixing ratio of (A) and (B) is (A)/(B)=99/1-70/30 (wt % ratio), and the concentration of (A) or (C) is the highest of all the surfactant ingredients in the composition:
(A) a sulfate type anionic surfactant,
(B) an N-acyl-α-alanine represented by the formula (I): 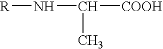
wherein R is a saturated or unsaturated acyl group having 8 to 26 carbon atoms, or a salt thereof,
(C) an amphoteric surfactant.

(2) A cleansing composition for skin or hair, which contains the following (A), (B) and (D), wherein the mixing ratio of (A) and (B) is (A)/(B)=99/1-70/30 (wt % ratio) and the concentration of (A) is the highest of all the surfactant ingredients in the composition:
(A) a sulfate type anionic surfactant,
(B) an N-acyl-α-alanine represented by the formula (I): 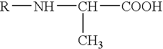
wherein R is a saturated or unsaturated acyl group having 8 to 26 carbon atoms, or a salt thereof,
(D) a cationic polymer.

(3) A cleansing composition for skin or hair, which contains the following (A), (B) and (E), wherein the mixing ratio of (A) and (B) is (A)/(B)=99/1-70/30 (wt % ratio), and the concentration of (A) is the highest of all the surfactant ingredients in the composition,
(A) a sulfate type anionic surfactant,
(B) an N-acyl-α-alanine represented by the formula (I): 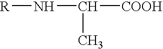
wherein R is a saturated or unsaturated acyl group having 8 to 26 carbon atoms, or a salt thereof,
(E) a silicone compound.

(4) A cleansing composition for skin or hair, which contains the following (A), (B) and (F), wherein the mixing ratio of (A) and (B) is (A)/(B)=99/1-70/30 (wt % ratio) and the concentration of (A) is the highest of all the surfactant ingredients in the composition:
(A) a sulfate type anionic surfactant,
(B) an N-acyl-α-alanine represented by the formula (1): 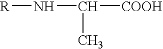
wherein R is a saturated or unsaturated acyl group having 8 to 26 carbon atoms, or a salt thereof,
(F) an oily material.

(5) A cleansing composition for skin or hair, which contains the following (A) and (B), and two or more kinds selected from the following (C), (D), (E) and (F), wherein the mixing ratio of (A) and (B) is (A)/(B)=99/1-70/30 (wt % ratio) and the concentration of (A) or (C) is the highest of all the surfactant ingredients in the composition:
(A) a sulfate type anionic surfactant,
(B) an N-acyl-α-alanine represented by the formula (1): 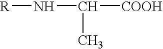
wherein R is a saturated or unsaturated acyl group having 8 to 26 carbon atoms, or a salt thereof,
(C) an amphoteric surfactant,
(D) a cationic polymer,
(E) a silicone compound,
(F) an oily material.

(6) A cleansing composition for skin or hair, which is obtained by adding (G) a fatty acid or a salt thereof to any one of the cleansing compositions of (1)-(5).

(7) The cleansing composition of any of (1)-(6), wherein (B) an N-acyl-α-alanine or a salt thereof is an N-acyl-L-alanine or a salt thereof.

(8) The cleansing composition of any of (1)-(7), which is a liquid cleansing composition.

(9) A cleansing agent for skin or hair, which contains a cleansing composition of any of (1)-(7).

(10) A method of cleaning a body surface in a subject in need thereof comprising contacting the surface in need of such treatment with a cleansing composition of any of (1)-(9), lathering said cleansing composition, rinsing said composition from said surface, and drying said surface. In the method of (10), the surface in need of such treatment is skin or hair.

The above objects highlight certain aspects of the invention. Additional objects, aspects and embodiments of the invention are found in the following detailed description of the invention.

### DETAILED DESCRIPTION OF THE INVENTION

Unless specifically defined, all technical and scientific terms used herein have the same meaning as commonly understood by a skilled artisan in enzymology, biochemistry, cellular biology, molecular biology, cosmetics, and the medical sciences.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

Ingredient (A) of the cleansing composition of the present invention is a sulfate-type anionic surfactant. Known sulfate-type anionic surfactants can be used without any limitation. Of these, alkyl sulfate, ether sulfate type anionic surfactants and the like are preferable.

The above-mentioned alkyl sulfate type anionic surfactant is an ester salt of a straight chain or branched chain, saturated or unsaturated higher alcohol having 8 to 22 carbon atoms and sulfuric acid and, for example, lauryl sulfate, myristyl sulfate, oleyl sulfate and the like can be mentioned.

The above-mentioned ether sulfate type anionic surfactant is an alkyleneoxide addition type of the aforementioned alkyl sulfate and, for example, polyoxyethylenelauryl ether sulfate, polyoxyethylene myristyl ether sulfate, polyoxyethylene oleoylether sulfate and the like can be mentioned.

As the basic component of these anionic surfactants, alkali metals such as sodium, potassium etc.; alkaline earth metals such as magnesium, calcium etc.; organic amines such as alkanolamine (e.g., monoethanolamine, diethanolamine, triethanolamine etc.), amino alcohol (e.g., 2-amino-2-methyl-1-propanol, 2-amino-2-methyl-1,3-propanediol etc.), basic amino acid (e.g., lysine, ornithine, arginine etc.) and the like; and inorganic amines such as ammonia etc.; and the like can be mentioned. These basic components can be used alone or in a combination of two or more kinds thereof.

In the present invention, one or more kinds of sulfate type anionic surfactants may be used for the ingredient (A). While the amount thereof to be added is appropriately determined according to the object product (i.e. specific use of cleansing composition), it is generally 1-60 wt %, preferably 3-15 wt %, more preferably 5-15 wt %, of the whole composition, from the viewpoints of lathering performance and lather amount.

Component (B) to be used in the present invention is an N-acyl-α-alanine or a salt thereof represented by the formula (I), and encompasses N-acyl-D-alanine, N-acyl-L-alanine, N-acyl-DL-alanine and a mixture of two or more kinds selected from these.

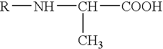

The saturated or unsaturated acyl group, which may be found in formula (1), has 8 to 26 carbon atoms for R in the formula (I) may be branched or straight chain and, for example, capryloyl group, caprinoyl group, lauroyl group, myristoyl group, palmitoyl group, stearoyl group, oleoyl group and the like can be mentioned.

In the present invention, the compound of the formula (I) may be a single compound wherein the acyl groups represented by R in the formula are the same, or a mixture of compounds wherein the acyl groups are different. As such mixture, compounds having an acyl group derived from naturally obtained mixed fatty acids such as coconut oil fatty acid, tallow fatty acid, hydrogenated tallow fatty acid, castor oil fatty acid, olive oil, palm oil fatty acid and the like or synthetically obtained fatty acids (including branched chain fatty acid) and the like can be mentioned. In the compound of the formula (I), R in the formula is preferably an acyl group derived from a mixed fatty acid, wherein the acyl group (i.e., R) has 8-26, preferably 8-18 carbon atoms on average, and particularly preferable acyl group derived from a mixed fatty acid is an acyl group derived from coconut oil fatty acid.

The basic component of a salt of the compound of the formula (I) may be an alkali metal (e.g., sodium, potassium etc.), an alkaline earth metal (e.g., magnesium, calcium etc.), and a salt comprising an organic amine and the like as a basic component can be mentioned. Here, as the organic amine, for example, alkanolamines such as monoethanolamine, diethanolamine, triethanolamine and the like, amino alcohols such as 2-amino-2-methyl-1-propanol, 2-amino-2-methyl-1,3-propanediol and the like, basic amino acids such as lysine, ornithine, arginine and the like, and the like can be mentioned. The salt (basic component) is preferably alkali metal salt, alkanolamine, basic amino acids and the like in view of stability of the product form, high solubility, and easy availability thereof, alkali metal salt is more preferable in view of stability of the product form and the like, and a sodium salt is most preferable from the viewpoints of spreading of lather and lather retention.

Preferable specific examples of the compound of the formula (I) or a salt thereof include, for example, N-cocoyl-DL-alanine sodium salt, N-cocoyl-L-alanine sodium salt, N-cocoyl-DL-alanine potassium salt, N-cocoyl-L-alanine potassium salt, N-cocoyl-DL-alanine triethanolamine salt, N-cocoyl-L-alanine triethanolamine salt, N-cocoyl-DL-alanine lysine salt, N-cocoyl-L-alanine lysine salt, N-cocoyl-DL-alanine arginine salt, N-cocoyl-L-alanine arginine salt, N-lauroyl-DL-alanine sodium salt, N-lauroyl-L-alanine sodium salt, N-lauroyl-DL-alanine potassium salt, N-lauroyl-L-alanine potassium salt, N-lauroyl-DL-alanine triethanolamine salt, N-lauroyl-L-alanine triethanolamine salt, N-myristoyl-DL-alanine sodium salt, N-myristoyl-L-alanine sodium salt, N-myristoyl-DL-alanine potassium salt, N-myristoyl-L-alanine potassium salt, N-myristoyl-DL-alanine triethanolamine salt, N-myristoyl-L-alanine triethanolamine salt, N-stearoyl-DL-alanine sodium salt, N-stearoyl-L-alanine sodium salt, N-stearoyl-DL-alanine potassium salt, N-stearoyl-L-alanine potassium salt, N-stearoyl-DL-alanine triethanolamine salt, and N-stearoyl-L-alanine triethanolamine salt.

In the present invention, N-acyl-α-alanine or a salt thereof (ingredient (B)) is preferably N-acyl-DL-alanine or a salt thereof or N-acyl-L-alanine or a salt thereof in view of easy availability, and use of N-acyl-L-alanine or a salt thereof is particularly preferable from the viewpoints of less residual feeling of cleansing agents during rinsing and the like.

From the foregoing, the most preferable N-acyl-α-alanine or a salt thereof (ingredient (B)) in the present invention is N-cocoyl-L-alanine Na salt.

In the present invention, N-acyl-α-alanine or a salt thereof (ingredient (B)) may be used alone or two or more kinds thereof may be used concurrently. While the amount thereof to be added is determined appropriately depending on the object product (i.e. specific use of cleansing composition) and is not particularly limited, it is generally 0.03-10 wt %, preferably 0.1-5 wt %, more preferably 0.1-3 wt %, of the whole composition.

In the cleansing composition of the present invention, it is important that the mixing ratio of the sulfate type anionic surfactant for ingredient (A) and N-acyl-α-alanine or a salt thereof for ingredient (B) be (A)/(B)=99/1-70/30 (ratio in wt %). When the mixing ratio is apart from this range and the amount of ingredient (A) is too high (the amount of ingredient (B) is too small), the feel of the skin and/or hair during rinsing or drying becomes poor, and when the amount of ingredient (A) is too small (the amount of ingredient (B) is too high), the basic performance of the cleansing agent becomes poor in terms of lather amount and lather retention, as well as the feel of the skin during drying becomes poor. In the present invention, the mixing ratio of ingredient (A) and ingredient (B) ((A)/(B)) is preferably 99/1-80/20 (wt % ratio), more preferably 95/5-80/20 (wt % ratio).

The amphoteric surfactant for component (C) to be used in the present invention is not particularly limited and known amphoteric surfactants can be used. Of these, carbobetaine type amphoteric surfactants, amidobetaine type amphoteric surfactants, sulfobetaine type amphoteric surfactants, hydroxysulfobetaine type amphoteric surfactants, amidosulfobetaine type amphoteric surfactants, phosphobetaine type amphoteric surfactants, imidazoline type amphoteric surfactants and the like are preferable, and amidobetaine type amphoteric surfactants are particularly preferable. Specific examples thereof include lauryldimethylaminoacetic acid betaine, coconut oil alkyl betaine, stearyldimethylamino acetic acid betaine, stearyldihydroxyethyl betaine, laurylamidopropyl betaine, cocamidopropyl betaine liquid, palm kernaloylamidopropyl betaine liquid, ricinoleoylamidopropyl betaine liquid, N-cocoyl-N-carboxyethyl-N-hydroxyethylethylenediamine salt, palm kernaloyl-N-carboxyethyl-N-hydroxyethylethylenediamine salt, 2-alkyl-N-carboxymethyl-N-hydroxyethylimidazolium betaine, undecyl-N-carboxymethylimidazolinium betaine, hydrochloric acid alkyldiaminoethylglycine liquid, bis(stearyl-N-hydroxyethylimidazoline)chloroacetic acid complex and the like.

Due to the concurrent use of ingredient (C) together with ingredient (A) and ingredient (B) in the present invention, the cleansing composition comprising the ingredients (A), (B) and (C) (cleansing composition of the first embodiment) shows preferable performance in a residual feeling during rinsing and a clarifying feeling after drying of the skin, as well as preferable performance in a residual feeling during rinsing and softness and lightness after drying of the hair.

The amphoteric surfactant for ingredient (C) may be used alone or two or more kinds thereof may be used concurrently. While the amount thereof to be added is determined appropriately depending on the object product (i.e. specific use of cleansing composition) and is not particularly limited, it is generally 0.5-15 wt %, preferably 2-10 wt %, of the whole composition.

In this composition (cleansing composition of the first embodiment), a composition ratio at which the concentration of ingredient (A) or ingredient (C) becomes the highest of all surfactant components in the composition is preferable from the viewpoints of the basic performance of cleansing agents such as lather amount and lather retention.

The cationic polymer for ingredient (D) to be used in the present invention is not particularly limited, and known cationic polymers can be used. Of these, chitin derivatives such as chitosan, partially hydrolyzed chitin, chitosan dl-pyrrolidonecarboxylic acid salt, succinylchitosan, hydroxypropylchitosan and the like; dimethyl diallylammonium chloride derivatives such as dimethyl diallylammonium chloride acrylamide copolymer, polydimethyl methylene piperidinium chloride and the like; cationic cellulose such as chlorinated O-[2-hydroxy-3-(trimethylammonio)propyl]hydroxyethylcellulose, chlorinated O-[2-hydroxy-3-(lauryl dimethylammonio)propyl]hydroxyethylcellulose and the like; cationic guargum such as chlorinated O-[2-hydroxy-3-(trimethylammonio)propyl]guargum and the like; methacrylic acid derivatives such as methacryloylethyldimethyl betaine methacryloylethyltrimethylammonium chloride methacrylic acid methoxypolyethylene glycol copolymer, methacryloylethyldimethyl betaine methacryloylethyltrimethylammonium chloride 2-hydroxyethyl methacrylate copolymer, vinylpyrrolidone-N,N-dimethylaminoethylmethacrylic acid copolymer diethyl sulfate salt and the like; vinylimidazolinium metachloride vinylpyrrolidone copolymer and the like are preferable, and cationic cellulose, cationic guargum and the like are particularly preferable.

Due to the concurrent use of ingredient (D) together with the ingredient (A) and ingredient (B) in the present invention, a cleansing composition containing the ingredients (A), (B) and (D) (cleansing composition of the second embodiment) particularly shows preferable performance in a frictional feeling during rinsing and a moist feeling after drying of the skin, as well as preferable performance in a frictional feeling during rinsing, a moist feeling after drying and softness and lightness after drying of the hair.

The cationic polymer for ingredient (D) may be used alone or two or more kinds thereof may be used concurrently. While the amount thereof to be used is determined appropriately depending on the object product (i.e. specific use of cleansing composition) and is not particularly limited, it is generally 0.01-2 wt %, preferably 0.01-0.5 wt %, of the whole composition.

In the cleansing composition containing the ingredients (A), (B) and (D) (cleansing composition of the second embodiment), it is important that the mixing ratio of the ingredient (A) and the ingredient (B) ((A)/(B)) be within the range defined above from the viewpoints mentioned above. It is also a preferred embodiment that the composition be one wherein the concentration of ingredient (A) is the highest of all the surfactant components, from the viewpoints of the basic performance of cleansing agents such as lather amount and lather retention. This composition preferably further contains the aforementioned ingredient (C). In this case, it is more preferable that the composition has a composition wherein the concentration of ingredient (A) or ingredient (C) is the highest of all the surfactant components from the viewpoints mentioned above.

In the present invention, the “surfactant” refers to the substances generally having a surface activation ability. Therefore, ingredient (B) is also a surfactant in the context of the present invention, not to mention ingredient (A) and ingredient (C), and fatty acid and a salt thereof (higher fatty acid soaps) for the below-mentioned ingredient (G) are also surfactants. In the composition of the present invention, moreover, surfactants such as acylamino acid salts (excluding “N-acyl-α-alanine salt” for ingredient (A)), anionic surfactants such as alkyletherphosphoric acid ester salts and the like, cationic surfactants such as alkyltrimethylammonium chloride, dialkyldimethylammonium chloride and the like, nonionic surfactants of polyoxyethylene type, polyhydric alcohol ester type, sugar ester type and the like, other polymer surfactants, natural surfactants and the like may be added besides these surfactants (ingredient (A), ingredient (B), ingredient (C), ingredient (G)).

In the present invention, by “the concentration of ingredient (A) (or ingredient (C)) is the highest of all the surfactant components” is meant that the concentration of ingredient (A) (or ingredient (C)) is the highest (content is high) of all the surfactant components even when the above-mentioned optional surfactants are contained.

The silicone compound to be used in the present invention for ingredient (E) is not particularly limited and known silicone compounds can be used. Of those, silicone resin, methylphenylpolysiloxane, methylpolysiloxane, octamethylcyclotetrasiloxane, dimethylsiloxane methyl(polyoxyethylene)siloxane copolymer, decamethylcyclopentasiloxane, dimethylsiloxane methyl(polyoxyethylene-polyoxypropylene)siloxane copolymer, methylhydrogenepolysiloxane, dodecamethylcyclohexasiloxane, methyl polycyclosiloxane, dimethylsiloxane methyl stearyxysiloxane copolymer, methyl polysiloxane emulsion, octamethyl trisiloxane, cyclic silicone resin, high polymerization methylpolysiloxane, tetradecamethylhexasiloxane, trimethylsiloxysilicate, polyether modified organopolysiloxane, fluoroalkylpolyoxyalkylene co-modified organopolysiloxane, alkyl modified organopolysiloxane, terminal modified organopolysiloxane, fluorine modified organopolysiloxane, amodimethicone, amino modified organopolysiloxane, silicone gel, acrylic silicone, trimethylsiloxysilicate, silicone RTV rubber and the like are preferable, and methylpolysiloxane, high polymerization methylpolysiloxane, decamethylcyclopentasiloxane and amodimethicone are particularly preferable.

Due to the concurrent use of ingredient (E) together with ingredient (A) and ingredient (B) in the present invention, a cleansing composition containing the ingredients (A), (B) and (E) (cleansing composition of the third embodiment) shows preferable performance in a frictional feeling and a residual feeling during rinsing of the skin, as well as preferable performance in a frictional feeling during rinsing and softness and lightness after drying of the hair.

The silicone compound for ingredient (E) may be used alone or two or more kinds thereof may be used concurrently. While the amount thereof to be added is determined appropriately depending on the object product (i.e. specific use of cleansing composition) and is not particularly limited, it is generally 0.01-20 wt %, preferably 0.1-10 wt %, more preferably 0.1-3 wt %, of the whole composition.

In the cleansing composition containing the ingredients (A), (B) and (E) (cleansing composition of the third embodiment), too, it is important that the mixing ratio of the ingredient (A) and the ingredient (B) ((A)/(B)) be within the range defined above from the viewpoints mentioned above. It is also a preferable embodiment that the composition is such that the concentration of ingredient (A) is the highest of all the surfactant components. This composition preferably further contains the aforementioned ingredient (C). In this case, it is more preferable that the concentration of ingredient (A) or ingredient (C) in the composition is the highest of all the surfactant components from the viewpoints mentioned above.

Ingredient (F) to be used in the present invention is an oily material, wherein the oily compound is any except the silicone compound for the aforementioned ingredient (E). Examples of suitable oily materials include, fats and oils containing, as a main component, triglyceride such as olive oil and castor oil, waxes such as carnauba wax, candelilla wax and jojoba oil, hydrocarbons such as liquid paraffin, petrolatum and squalene, higher fatty acids such as lauric acid, myristic acid, palmitic acid, stearic acid and isostearic acid, higher alcohols such as lauryl alcohol, myristyl alcohol, cetyl alcohol, stearyl alcohol, isostearyl alcohol and 2-octyldodecanol, ester oils such as isopropyl myristate, 2-octyldodecyl myristate and cetyl 2-ethylhexanate, and the like can be mentioned. Of these, higher alcohols and the like are preferable.

Due to the concurrent use of ingredient (F) together with ingredient (A) and ingredient (B) in the present invention, a cleansing composition containing the ingredients (A), (B) and (F) (cleansing composition of the fourth embodiment) shows preferable performance in a residual feeling during rinsing and a moist feeling after drying of the skin, as well as preferable performance in a residual feeling and a residual feeling during rinsing and a moist feeling after drying of the hair.

The oily material for ingredient (F) may be used alone or two or more kinds thereof may be used concurrently. While the amount thereof to be added is determined appropriately depending on the object product (i.e. specific use of cleansing composition) and is not particularly limited, it is generally 0.01-10 wt %, preferably 0.1-5 wt %, more preferably 0.1-2 wt %, of the whole composition.

In the cleansing composition containing the ingredients (A), (B) and (F) (cleansing composition of the fourth embodiment), too, it is important that the mixing ratio of the ingredient (A) and the ingredient (B) ((A)/(B)) be within the range defined above from the viewpoints mentioned above. It is a preferred embodiment that the composition has a concentration of ingredient (A), which is the highest of all the surfactant components. This composition preferably further contains the aforementioned ingredient (C). In this case, it is more preferable that the composition has a concentration of ingredient (A) or ingredient (C) which is the highest of all the surfactant components from the viewpoints mentioned above.

The cleansing composition of the present invention more preferably has a composition having a constitution (composition) composing not less than 2 of the constitutions (compositions) of the compositions of the aforementioned first to fourth embodiments. With such composition, more preferable results can be obtained.

With the aim of further improving the basic performance of cleansing agents, such as lathering performance and lather retention, and further reducing a residual feeling during rinsing of the skin and/or hair, the cleansing composition of the present invention may further contain a fatty acid salt or a salt thereof (higher fatty acid soaps)(=ingredient (G)).

The fatty acid or a salt thereof for ingredient (G) is a straight chain or branched chain, saturated or unsaturated fatty acid or a salt thereof, and comprises single compounds having the same carbon number or a mixture of compounds having different carbon numbers. As the basic component in the case of salt, alkali metals such as sodium, potassium and the like, alkaline earth metals such as magnesium, calcium and the like, organic amines such as monoethanolamine, diethanolamine, triethanolamine, 2-amino-2-methyl-1-propanol, 2-amino-2-methyl-1,3-propanediol and the like, inorganic amines such as ammonia and the like, basic amino acids such as lysine, ornithine, arginine and the like, and the like can be mentioned, with preference given to alkali metal. The fatty acid or a salt thereof preferably has 8-26, particularly preferably 8-18, carbon atoms (average carbon number in the case of a mixture), and lauric acid, myristic acid, palmitic acid, stearic acid, coconut oil fatty acid, hydrogenated tallow fatty acid, palm oil fatty acid, hydrogenated palm oil fatty acid, safflower oil fatty acid, tallow fatty acid, behenic acid, oleic acid, isostearic acid and the like and salts of these can be specifically mentioned. Of these, lauric acid (salt), myristic acid (salt), palmitic acid (salt) and coconut oil fatty acid (salt) are preferable.

The fatty acid or a salt thereof (higher fatty acid soaps) for ingredient (G) may be used alone or two or more kinds thereof may be used concurrently. While the amount thereof to be added is determined appropriately depending on the object product (i.e. specific use of cleansing composition) and is not particularly limited, it is generally 0.001-2 wt %, preferably 0.01-0.5 wt %, of the whole composition.

In a cleansing composition of an embodiment further containing the ingredient (G), too, it is important that the composition have a composition wherein the concentration of ingredient (A) is the highest of all the surfactant components (including ingredient (G)) from the viewpoints mentioned above. In addition, a composition containing ingredient (C) together with ingredient (A) is preferable. In this case, it is more preferable that the composition has a concentration of ingredient (A) or ingredient (C), which is the highest of all the surfactant components (including ingredient (G)) from the viewpoints mentioned above.

In addition to the foregoing components, the cleansing composition of the present invention can also contain components such as oil, powder (pigment, dye, resin), fluorine compound, resin, adhesive, polymer, preservative, flavor, UV absorber, moisturizer, polyhydric alcohol, physiologically active component, salts, solvent, antioxidant, antibacterial agent, adiaphoretic, chelating agent, neutralization agent, pH adjusting agent and the like, which are generally used for cosmetic agents, within the range the object (effect) of the present invention is not inhibited.

While the cleansing composition of the present invention can be used upon adjusting same to compositions in a wide range from weak acidic to weak basic, it is preferably processed into a weak acidic composition of pH 5.0-6.8, from the viewpoints of reduction of irritation to the skin and hair and the like.

The dosage (form) of the cleansing composition of the present invention is not particularly limited, and any dosage (form) of liquid, paste, gel, solid, powder and the like can be employed. From the viewpoints of quick lathering and the like, however, it is preferably a liquid.

The cleansing composition of the present invention can be used as various skin cleansing agents such as various hair cleansing agents (e.g., shampoo, conditioning shampoo etc.), face cleansing agent, makeup removing agents, cleansing cream, body shampoo, hand soap, solid soap, shaving foam, shaving cream and the like, and can be also used as toothpaste and the like.

As is evident from the following Examples, the present invention provides a cleansing composition that lathers quickly during cleansing, is superior in the basic performance of cleansing agents such as lather amount, spreading of lather and lather retention, free of a residual feeling or a frictional feeling during rinsing, and gives, after drying, a moist feeling and a clarifying feeling after use to the skin and a moist feeling and simultaneous softness and lightness to the hair.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

### Production Example 1

### Synthesis of N-cocoyl-L-alanine sodium salt

Water (167 g) was added to L-alanine (30.0 g), and cocoyl chloride (73.68 g) and 27 wt % aqueous NaOH solution were simultaneously added dropwise over 1 hr with stirring while maintaining pH of 10.98-11.02 and reaction temperature of 33-37° C. After aging for 1 hr, the reaction mixture was heated and 75 wt % sulfuric acid was added to adjust the pH to 2.0. The mixture was further heated to 80° C., stirred for about 10 min and incubated without mixing for 10 min to allow partitioning into an organic layer and an aqueous layer. The aqueous layer (about 200 g) was removed from the bottom part of a container. Hot water was added to the remaining organic layer, and the mixture was heated again to 80° C., stirred for about 10 min and incubated without mixing for 10 min. The partitioned aqueous layer was removed to give an organic layer. A 27% aqueous sodium hydroxide solution and water were added to the organic layer to adjust the pH to 6.8, whereby an object product (N-cocoyl-L-alanine sodium salt) was obtained.

### Production Example 2

### Synthesis of N-cocoyl-DL-alanine sodium salt

In the same manner as in Production Example 1 except that DL-alanine was used instead of L-alanine, and, as such, the object product (N-cocoyl-DL-alanine sodium salt) was obtained.

### Examples 1-10, Comparative Examples 1-12

The cleansing compositions (Examples 1-10, Comparative Examples 1-12) having the compositions shown in the following Table 1 were prepared, and subjected to the following evaluation tests. In addition to the components listed in the table for each composition, the balance of the composition was distilled water, and the amount (numerical value in Tables) of each added component described in the Tables is weight fraction (%) when the whole composition is 100.

Evaluation Tests—

(i) Evaluation of the Basic Performance of the Compositions of Examples 1-10, Comparative Examples 1-12 to Serve as Cleansing Agents, Including Lathering Performance, Lather Amount, Lather Spreading, Lather Retention and the Like

Five professional panelists performed hand-washing evaluation. For evaluation, the average point obtained by the following grade table of not less than 4.1 was marked with ⊙, 3.5-4.0 was marked with ◯, 3.0-3.4 was marked with Δ, and not more than 2.9 was marked with x.

Lathering Performance
5: quick
4: rather quick
3: average
2: rather late
1: late

Lather Amount
5: thick
4: rather thick
3: average
2: rather insufficient
1: insufficient

Spreading of Lather
5: fine
4: rather fine
3: average
2: rather unpreferable
1: unpreferable

Lather Retention
5: fine
4: rather fine
3: average
2: rather unpreferable
1: unpreferable
   (ii) Functional Evaluation by Hand Washing Test

In the hand washing test, a residual feeling and a frictional feeling during rinsing, and a moist feeling and a clarifying feeling after drying were also evaluated by five professional panelists.

In the hair cleansing test, a residual feeling and a frictional feeling during rinsing and a moist feeling, as well as softness and lightness after drying, were evaluated by five professional panelists.

The evaluation was based on 5 levels of 1 point (bad) to 5 points (fine) and the average point of five panellers was calculated, wherein the average of not less than 4.1 was marked with ⊙, 3.5-4.0 was marked with ◯, 3.0-3.4 was marked with ▴, and not more than 2.9 was marked with x.

TABLE 1

| | | | | | | | | | | | Comparative | | | |
|---|---|---|---|---|---|---|---|---|---|---|---|---|---|---|
| | Examples | | | | | | | | | | Examples | | | |
| ingredients added | 1 | 2 | 3 | 4 | 5 | 6 | 7 | 8 | 9 | 10 | 1 | 2 | 3 | 4 |
| sodium polyoxyethylene lauryl | 10 | 10 | 10 | 10 | 10 | 10 | 10 | 10 | 10 | 10 | 10 | 8 | 10 | 10 |
| ether sulfate *1 | | | | | | | | | | | | | | |
| N-cocoyl-DL-alanine sodium | | | | | 2 | 2 | 2 | | | | | | | |
| salt | | | | | | | | | | | | | | |
| N-cocoyl-L-alanine sodium | 2 | 2 | 2 | 2 | | | | 2 | 2 | 2 | 2 | 4 | | |
| salt | | | | | | | | | | | | | | |
| N-cocoyl glutamic acid sodium | | | | | | | | | | | | | 2 | 2 |
| salt *2 | | | | | | | | | | | | | | |
| N-cocoyl sarcosine sodium salt *3 | | | | | | | | | | | | | | |
| N-cocoyl-N-methyl-β-alanine | | | | | | | | | | | | | | |
| sodium salt *4 | | | | | | | | | | | | | | |
| cocamidepropylbetaine *5 | 3 | | | | 3 | | | 3 | 3 | 3 | | | 3 | |
| cationic cellulose *6 | | 0.2 | | | | 0.2 | | 0.2 | | | | 0.2 | | 0.2 |
| dimethicone *7 | | | 0.8 | | | | 0.8 | | | | | | | |
| lauryl alcohol/myristyl alcohol *8 | | | | 0.7 | | | | | 0.7 | | | | | |
| sodium laurate | | | | | | | | | | 0.2 | | | | |
| (basic performance) | | | | | | | | | | | | | | |
| lather | ⊙ | ⊙ | ⊙ | ⊙ | ◯ | ◯ | ◯ | ⊙ | ⊙ | ⊙ | ⊙ | ⊙ | ▴ | ▴ |
| lather amount | ⊙ | ⊙ | ⊙ | ⊙ | ⊙ | ⊙ | ⊙ | ⊙ | ⊙ | ⊙ | ⊙ | ▴ | ◯ | ◯ |
| spreading of lather | ⊙ | ⊙ | ⊙ | ⊙ | ⊙ | ⊙ | ⊙ | ⊙ | ⊙ | ⊙ | ⊙ | ◯ | ⊙ | ⊙ |
| lather retention | ⊙ | ⊙ | ◯ | ◯ | ⊙ | ◯ | ◯ | ⊙ | ⊙ | ⊙ | ⊙ | ▴ | ◯ | ◯ |
| (skin) | | | | | | | | | | | | | | |
| residual feeling during rinsing | ⊙ | ◯ | ⊙ | ⊙ | ▴ | ▴ | ▴ | ◯ | ⊙ | ⊙ | ▴ | ◯ | ▴ | X |
| frictional feeling during rinsing | ◯ | ⊙ | ⊙ | ⊙ | ◯ | ⊙ | ⊙ | ⊙ | ⊙ | ◯ | ◯ | ◯ | ◯ | ⊙ |
| moist feeling after drying | ◯ | ⊙ | ◯ | ⊙ | ◯ | ⊙ | ◯ | ⊙ | ⊙ | ◯ | ▴ | ⊙ | ⊙ | ⊙ |
| clarifying feeling after drying | ⊙ | ◯ | ⊙ | ◯ | ⊙ | ◯ | ⊙ | ⊙ | ⊙ | ⊙ | ◯ | ▴ | X | X |
| (hair) | | | | | | | | | | | | | | |
| residual feeling during rinsing | ⊙ | ◯ | ⊙ | ⊙ | ◯ | ▴ | ◯ | ◯ | ⊙ | ⊙ | ◯ | ◯ | ▴ | X |
| frictional feeling during rinsing | ◯ | ⊙ | ⊙ | ⊙ | ◯ | ⊙ | ◯ | ⊙ | ⊙ | ◯ | ▴ | ◯ | ◯ | ⊙ |
| moist feeling after drying | ◯ | ⊙ | ◯ | ⊙ | ◯ | ⊙ | ◯ | ⊙ | ⊙ | ◯ | ▴ | ◯ | ⊙ | ⊙ |
| softness and lightness after | ⊙ | ⊙ | ⊙ | ⊙ | ◯ | ◯ | ◯ | ⊙ | ⊙ | ⊙ | ◯ | ⊙ | ▴ | X |
| drying | | | | | | | | | | | | | | |

| | Comparative Examples | | | | | | | |
|---|---|---|---|---|---|---|---|---|
| ingredients added | 5 | 6 | 7 | 8 | 9 | 10 | 11 | 12 |
| sodium polyoxyethylene lauryl | 10 | 10 | 10 | 10 | 10 | 10 | 10 | 10 |
| ether sulfate *1 | | | | | | | | |
| N-cocoyl-DL-alanine sodium | | | | | | | | |
| salt | | | | | | | | |
| N-cocoyl-L-alanine sodium | | | | | | | | |
| salt | | | | | | | | |
| N-cocoyl glutamic acid sodium | 2 | | | | | | | |
| salt *2 | | | | | | | | |
| N-cocoyl sarcosine sodium salt *3 | | 2 | 2 | 2 | | | 2 | 2 |
| N-cocoyl-N-methyl-β-alanine | | | | | 2 | 2 | | |
| sodium salt *4 | | | | | | | | |
| cocamidepropylbetaine *5 | | 3 | | | | | 3 | 3 |
| cationic cellulose *6 | | | 0.2 | | 0.2 | | 0.2 | 0.2 |
| dimethicone *7 | 0.8 | | | 0.8 | | | | |
| lauryl alcohol/myristyl alcohol *8 | | | | | | 0.7 | | 0.7 |
| sodium laurate | | | | | | | | |
| (basic performance) | | | | | | | | |
| lather | ▴ | ◯ | ◯ | ◯ | ◯ | ◯ | ◯ | ◯ |
| lather amount | ◯ | ⊙ | ⊙ | ⊙ | ⊙ | ⊙ | ⊙ | ⊙ |
| spreading of lather | ⊙ | ⊙ | ⊙ | ⊙ | ⊙ | ⊙ | ⊙ | ⊙ |
| lather retention | ◯ | ⊙ | ◯ | ▴ | ◯ | ▴ | ⊙ | ⊙ |
| (skin) | | | | | | | | |
| residual feeling during rinsing | ▴ | ▴ | ▴ | ▴ | ▴ | ▴ | ▴ | ▴ |
| frictional feeling during rinsing | ◯ | ▴ | ◯ | ◯ | ◯ | ◯ | ◯ | ◯ |
| moist feeling after drying | ⊙ | ▴ | ▴ | ▴ | ▴ | ◯ | ▴ | ◯ |
| clarifying feeling after drying | ▴ | ◯ | ◯ | ◯ | ◯ | ▴ | ◯ | ▴ |
| (hair) | | | | | | | | |
| residual feeling during rinsing | ▴ | ◯ | ▴ | ▴ | ▴ | ▴ | ▴ | ▴ |
| frictional feeling during rinsing | ◯ | ▴ | ⊙ | ▴ | ⊙ | ◯ | ◯ | ◯ |
| moist feeling after drying | ◯ | ▴ | ▴ | ◯ | ▴ | ◯ | ◯ | ◯ |
| softness and lightness after | X | ▴ | ▴ | ▴ | ▴ | ▴ | ▴ | ▴ |
| drying | | | | | | | | |

In the Table,
*1: “Emarl” 20C manufactured by Kao Corporation
*2: “Amisoft” CS-22 manufactured by Ajinomoto Co., Inc.
*3: “Soypon” SC manufactured by Kawaken Fine Chemicals Co., Ltd.
*4: “Alanon” ACE manufactured by Kawaken Fine Chemicals Co., Ltd.
*5: “Softazoline” CPB manufactured by Kawaken Fine Chemicals Co., Ltd.
*6: Polymer JR-400 manufactured by Amerchol
*7: SH200 (10000) manufactured by Toray-Dow Corning Corporation
*8: lauryl alcohol/myristyl alcohol = 2/1 (wt %)

As is evident from Table 1, the cleansing compositions of Examples quickly lather during cleansing, are superior in lather amount, lather spreading, and lather retention, do not give a residual feeling or a frictional feeling during rinsing of the skin and hair, give a moist feeling and a clarifying feeling to the skin after drying, and give a moist feeling and simultaneously softness and lightness to hair, as compared to the compositions of Comparative Examples.

### Examples 11-14

The liquid hand soap (Example 11), conditioning shampoo (Example 12), body shampoo (Example 13) and shampoo (Example 14) were prepared by conventional methods according to the formulations shown in the following Tables. Each of these formulation are fine in the basic evaluation of lathering performance, lather amount, spreading of lather and lather retention, and also superior in feeling of use. The amount (numerical value in Table) of each added component described in each Table is weight fraction (%) when the whole composition is 100.

TABLE 2
(liquid hand soap)

| | Amount added |
|---|---|
| Substances added | (wt %) |
| polyoxyethylene laurylether sulfate | 8 |
| lauryl sulfate | 2 |
| N-cocoyl-L-alanine sodium salt | 2 |
| sodium chloride | 2 |
| glycol distearate | 1 |
| dimethicone | 0.8 |
| cetanol | 0.5 |
| cocamide MEA | 1 |
| flavor | 0.2 |
| sodium citrate | 0.2 |
| guarhydroxy propyl trimonium chloride | 0.2 |
| ammonium xylenesulfate | 0.1 |
| olefin oligomer | 0.1 |
| benzoate | 0.1 |
| edetate | 0.1 |
| tri(capryl/caprylic acid) trimethylolpropane | 0.1 |
| laureth-4 | 0.2 |
| citric acid | 0.1 |
| panthenol | 0.1 |
| panthenyl ethyl | 0.1 |
| methylchloroisothiazolinon | 0.2 |
| methylisothiazolinon | 0.2 |
| stearyl alcohol | 0.1 |
| water | remaining |
| | portion |

TABLE 3
(Conditioning shampoo)

| | Amount added |
|---|---|
| Substances added | (wt %) |
| polyoxyethylene laurylether sulfate | 9 |
| sodium chloride | 2 |
| N-cocoyl-DL-alanine triethanolamine salt | 2 |
| cocamidepropyl betaine | 2 |
| lauryl sulfate | 1 |
| dimethicone | 0.8 |
| glycol distearate | 1 |
| cocamide MEA | 1 |
| lauroyl sarcosinate | 1 |
| ammonium xylenesulfate | 0.5 |
| cetanol | 0.5 |
| flavor | 0.2 |
| polyquaternium-10 | 0.2 |
| disodium phosphate | 0.1 |
| stearyl alcohol | 0.1 |
| sodium phosphate | 0.2 |
| edetate | 0.05 |
| panthenyl ethyl | 0.1 |
| panthenol | 0.1 |
| methylchloroisothiazolinon | 0.01 |
| methylisothiazolinon | 0.01 |
| benzyl alcohol | 0.05 |
| benzoate | 0.1 |
| benzoic acid | 0.1 |
| water | remaining |
| | portion |

TABLE 4
(body shampoo)

| | Amount added |
|---|---|
| Substances added | (wt %) |
| polyoxyethylene laurylether sulfate | 7 |
| cocamidepropyl betaine | 3 |
| glycol distearate | 2 |
| N-cocoyl-L-alanine sodium salt | 2 |
| sodium chloride | 1 |
| dimethiconol | 0.8 |
| arginine | 0.5 |
| oleic acid | 0.1 |
| sodium lauroyl hydrolyzed silk | 0.2 |
| silk extract | 0.1 |
| perfluoropolymethylisopropyl | 0.1 |
| guarhydroxypropyl trimonium chloride | 0.1 |
| decyl glucoside | 1 |
| PPG-9 | 0.4 |
| citric acid | 0.2 |
| cocamide MEA | 0.3 |
| laureth-10 | 0.2 |
| dodecylbenzensulfonic acid TEA | 0.5 |
| DPG | 0.2 |
| ethanol | 0.1 |
| salicylate | 0.2 |
| paraben | 0.2 |
| edetate | 0.05 |
| dibuthylhydroxytoluene | 0.02 |
| benzoate | 0.01 |
| flavor | 0.1 |
| water | remaining |
| | portion |

TABLE 5
(shampoo)

| | Amount added |
|---|---|
| Substances added | (wt %) |
| polyoxyethylene lauryl ether sulfate | 6 |
| cocamide DEA | 2 |
| sodium cocoamphoacetate | 1 |
| polyoxyethylene laurylether sulfate triethanolamine | 4 |
| N-cocoyl-L-alanine sodium salt | 2 |
| DPG | 1 |
| glycol distearate | 2 |
| sorbitol | 0.5 |
| macadamia nut fatty acid phytosteryl | 0.5 |
| hydrolyzed keratin | 0.2 |
| lecithin | 0.2 |
| dimethicone | 0.7 |
| cationic hydroxyl ethylcellulose-2 | 0.2 |
| citric acid | 0.2 |
| edetate | 0.05 |
| sodium olefin (C14-16) sulfonate | 1 |
| laureth-25 | 1 |
| hydrochloric acid TEA | 0.5 |
| BG | 1 |
| triethanol amine | 0.2 |
| benzoate | 0.2 |
| paraben | 0.2 |
| flavor | 0.15 |
| water | remaining |
| | portion |

Numerous modifications and variations on the present invention are possible in light of the above teachings. It is, therefore, to be understood that within the scope of the accompanying claims, the invention may be practiced otherwise than as specifically described herein.

1. A cleansing composition, which comprises:
(A) a sulfate type anionic surfactant,
(B) an N-acyl-α-alanine represented by the formula (I): 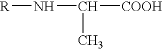
wherein R is a saturated or unsaturated acyl group having 8 to 26 carbon atoms, or a salt thereof,
(C) an amphoteric surfactant,
wherein the mixing ratio of (A) and (B) is (A)/(B)=99/1-70/30 (wt % ratio), and the concentration of (A) or (C) is the highest of all the surfactant ingredients in the composition.

2. A cleansing composition, which is obtained by adding (G) a fatty acid or a salt thereof to a cleansing compositions of claim 1.

3. The cleansing composition of claim 1, wherein (B) an N-acyl-α-alanine or a salt thereof is an N-acyl-L-alanine or a salt thereof.

4. The cleansing composition of claim 1, which is a liquid cleansing composition.

5. A method of cleaning a body surface in a subject in need thereof comprising contacting the surface in need of such treatment with a cleansing composition of claim 1, lathering said cleansing composition, rinsing said composition from said surface, and drying said surface.

6. The method of claim 5, wherein said surface in need of such treatment is skin.

7. The method of claim 5, wherein said surface in need of such treatment is hair.

8. A cleansing composition, which comprises:
(A) a sulfate type anionic surfactant,
(B) an N-acyl-α-alanine represented by the formula (I): 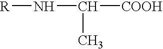
wherein R is a saturated or unsaturated acyl group having 8 to 26 carbon atoms, or a salt thereof,
(D) a cationic polymer,
wherein the mixing ratio of (A) and (B) is (A)/(B)=99/1-70/30 (wt % ratio) and the concentration of (A) is the highest of all the surfactant ingredients in the composition.

9. A cleansing composition, which is obtained by adding (G) a fatty acid or a salt thereof to a cleansing compositions of claim 8.

10. The cleansing composition of claim 8, wherein (B) an N-acyl-α-alanine or a salt thereof is an N-acyl-L-alanine or a salt thereof.

11. The cleansing composition of claim 8, which is a liquid cleansing composition.

12. A method of cleaning a body surface in a subject in need thereof comprising contacting the surface in need of such treatment with a cleansing composition of claim 8, lathering said cleansing composition, rinsing said composition from said surface, and drying said surface.

13. The method of claim 12, wherein said surface in need of such treatment is skin.

14. The method of claim 12, wherein said surface in need of such treatment is hair.

15. A cleansing composition, which comprises:
(A) a sulfate type anionic surfactant,
(B) an N-acyl-α-alanine represented by the formula (1): 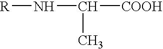
wherein R is a saturated or unsaturated acyl group having 8 to 26 carbon atoms, or a salt thereof,
(E) a silicone compound,
wherein the mixing ratio of (A) and (B) is (A)/(B)=99/1-70/30 (wt %), and the concentration of (A) is the highest of all the surfactant ingredients in the composition.

16. A cleansing composition, which is obtained by adding (G) a fatty acid or a salt thereof to a cleansing compositions of claim 15.

17. The cleansing composition of claim 15, wherein (B) an N-acyl-α-alanine or a salt thereof is an N-acyl-L-alanine or a salt thereof.

18. The cleansing composition of claim 15, which is a liquid cleansing composition.

19. A method of cleaning a body surface in a subject in need thereof comprising contacting the surface in need of such treatment with a cleansing composition of claim 15, lathering said cleansing composition, rinsing said composition from said surface, and drying said surface.

20. The method of claim 19, wherein said surface in need of such treatment is skin.

21. The method of claim 19, wherein said surface in need of such treatment is hair.

22. A cleansing composition, which comprises:
(A) a sulfate type anionic surfactant,
(B) an N-acyl-α-alanine represented by the formula (I): 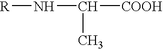
wherein R is a saturated or unsaturated acyl group having 8 to 26 carbon atoms, or a salt thereof,
(F) an oily material,
wherein the mixing ratio of (A) and (B) is (A)/(B)=99/1-70/30 (wt % ratio) and the concentration of (A) is the highest of all the surfactant ingredients in the composition.

23. A cleansing composition, which is obtained by adding (G) a fatty acid or a salt thereof to a cleansing compositions of claim 22.

24. The cleansing composition of claim 22, wherein (B) an N-acyl-α-alanine or a salt thereof is an N-acyl-L-alanine or a salt thereof.

25. The cleansing composition of claim 22, which is a liquid cleansing composition.

26. A method of cleaning a body surface in a subject in need thereof comprising contacting the surface in need of such treatment with a cleansing composition of claim 22, lathering said cleansing composition, rinsing said composition from said surface, and drying said surface.

27. The method of claim 26, wherein said surface in need of such treatment is skin.

28. The method of claim 26, wherein said surface in need of such treatment is hair.

29. A cleansing composition, which comprises the following (A) and (B), and two or more kinds selected from the following (C), (D), (E) and (F):
(A) a sulfate type anionic surfactant,
(B) an N-acyl-α-alanine represented by the formula (I): 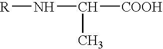
wherein R is a saturated or unsaturated acyl group having 8 to 26 carbon atoms, or a salt thereof,
(C) an amphoteric surfactant,
(D) a cationic polymer,
(E) a silicone compound,
(F) an oily material,
wherein the mixing ratio of (A) and (B) is (A)/(B)=99/1-70/30 (wt % ratio) and the concentration of (A) or (C) is the highest of all the surfactant ingredients in the composition.

30. A cleansing composition, which is obtained by adding (G) a fatty acid or a salt thereof to a cleansing compositions of claim 29.

31. The cleansing composition of claim 29, wherein (B) an N-acyl-α-alanine or a salt thereof is an N-acyl-L-alanine or a salt thereof.

32. The cleansing composition of claim 29, which is a liquid cleansing composition.

33. A method of cleaning a body surface in a subject in need thereof comprising contacting the surface in need of such treatment with a cleansing composition of claim 29, lathering said cleansing composition, rinsing said composition from said surface, and drying said surface.

34. The method of claim 33, wherein said surface in need of such treatment is skin.

35. The method of claim 33, wherein said surface in need of such treatment is hair.
